(19) United States
(12) Patent Application Publication
(10) Pub. No.: US 2011/0098292 A1
(43) Pub. Date: Apr. 28, 2011

(54) New Compounds Useful for Treating CNS Disorders

(75) Inventors: Jonas Malmstrom, Sodertalje (SE); Jenny Viklund, Sodertalje (SE)

(73) Assignee: AstraZeneca AB, Sodertalje (SE)

(21) Appl. No.: 11/571,184

(22) PCT Filed: Jun. 29, 2005
(86) PCT No.: PCT/SE05/01033

§ 371 Date: May 3, 2007

(30) Foreign Application Priority Data
Jul. 2, 2004 (SE) 0401716-6

(51) Int. Cl. A61K 31/428; C07D 417/14; A61K 31/4439; A61K 31/5377; C07D 417/04; A61P 25/30; A61P 9/10; A61P 25/28; A61P 25/16; A61P 25/00

(52) U.S. Cl. 514/233.8; 548/180; 514/373; 546/270.1; 514/338; 544/131; 548/178

(57) ABSTRACT

The present invention provides new compounds of formula (I) as well as a process for their preparation and new intermediates used therein, pharmaceutical formulations containing said therapeutically active compounds and to the use of said active compounds in therapy.

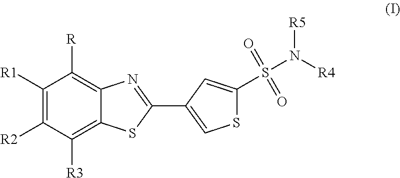

### FIELD OF THE INVENTION

The present invention relates to new compounds of formula I, as a free base or a pharmaceutically acceptable salt thereof, to pharmaceutical formulations containing said compounds and to the use of said compounds in therapy. The present invention further relates the process for the preparation of compounds of formula I and to new intermediates prepared therein.

An object of the invention is to provide compounds of formula I for therapeutic use, especially compounds that are useful for the prevention and/or treatment of conditions associated with cyclin-dependent kinase 5 (cdk5) in mammals including man.

It is also an object of the invention to provide compounds with a therapeutic effect after is oral administration.

### BACKGROUND OF THE INVENTION

Cyclin-dependent kinase 5 (cdk5) is a prolin-directed serine/threonine kinase that is activated by one of two of its non-cylin partners called p35 and p39. Due to the restricted localization of the activators, cdk5 enzymatic activity is in large part restricted to post-mitotic neuronal precursors and mature neurons. The enzyme has relatively broad substrate specificity in CNS and has its functions prominently in the developing nervous system. It is connected to regulation of cytoskeletal and membrane dynamics through interaction with several of the actin- and mictrotubule-associated proteins including tau, and the neurofilament network (Smith & Tsai 2002, Maccioni et al, 2001).

Alzheimer's Disease (AD) Dementias, and Taupathies

AD is characterized by cognitive decline, cholinergic dysfunction and neuronal death, neurofibrillary tangles and senile plaques consisting of amyloid-β deposits. The sequence of these events in AD is unclear, but is believed to be related. Specific kinases, including cdk5, are able to selectively phosphorylate the microtubule associated protein tau (τ) in neurons at sites that are hyperphosphorylated in AD brains. Hyperphosphorylated protein τ has lower affinity for microtubules and accumulates as paired helical filaments (PHF), which are the main components that constitute neurofibrillary tangles and neuropil threads in AD brains. This results in depolymerization of microtubules, which leads to dying back of axons and neuritic dystrophy. Neurofibrillary tangles are consistently found in diseases such as AD, amyotrophic lateral sclerosis, parkinsonism-dementia of Gaum, corticobasal degeneration, dementia pugilistica and head trauma, Down's syndrome, postencephalatic parkinsonism, progressive supranuclear palsy, Niemann-Pick's Disease and Pick's Disease.

Cdk5 is one of the two defined tau protein kinases (TPKs), that originally were identified in microtubule protein fractions in rat or bovine brain extracts as being able to phoshorylate tau into a PHF state (Imahori et al. 1998). It was later purified and designated as TPKII, GSK3β being designated as TPKI (Omori et al 1991, Imahori et al. 1998). These two kinases phosphorylate PHF tau in human AD material at partly overlapping sites (Hanger et al. 1998, Morishima-Kawashima et al 1995). Furthermore an apparent increase of cdk5 immunoreactivity was found to overlap with that of hyperphosphorylated tau in the neocortical pyramidal neurons and cerebrellar neurons of AD and that the increase in immunostaining was particularly prominent in pretangle neurons indicating a relatively early involvement of the enzyme (Pei et al. 1998).

Furthermore, in AD patients, large increase in the activator p25 has been detected alongside withan increase in enzymatic activity (Patrick et al. 1999). This has been connected with presence of the calcium-dependent protease calpain that appears to be responsible for the processing of the activator p35 to p25 (Lee at al. 2002). P25 differs from p35 in stability and subcellular localization. The half-life of p25 is about 5-10 times that of p35 and this slow turnover rate may contribute to its accumulation in the AD brain. The cleavage also leads to the loss of a myristoylation connection to the cell membrane, which might put the enzyme in a better position to phosphorylate tau in the cytosol (Patrick et al.1999).

An additional piece of evidence pointing to a role of cdk5/p25 in pathogenesis of AD is the recent report on inducible p25 transgenic mice. These animals exhibit neuronal loss in the cortex and hippocampus, accompanied by forebrain atrophy and hyperphosphorylated aggregated tau that progressively developed into neurofibrillary pathology. (Cruz, J. C. et al (2003) Neuron 40 471-483). These findings lead to a strong suggestion to that aberrant cdk5 activity can lead to neurodegeneration and tau fibrillary pathology in vivo in absence of tau dysregulation or mutations.

Amyotrophic Lateral Sclerosis—ALS

Amyotrophic lateral sclerosis (ALS) is an adult-onset neurological disorder characterized by selective degeneration of motor neurons leading to paralysis and death within 3-5 years. Specific mutations in the gene for SOD1 (superoxide dismutase 1) have been identified as one of the causes of familial cases of ALS and transgene mice expressing one of these mutations have been used to study the disease mechanisms. Mislocalization and hyperactivation of cdk5 due to increased ratio of p25/p35 was observed in these animals leading to hyperphosphorylation of neurofilaments by the active cdk5 (Nguyen, M. D. et al (2001) Neuron 30, 135-147). This was suggested to be potentially connected to the death of motor neurons in ALS since in ALS patients, pathological accumulations of phosphorylated NF-H partially overlap with staining for cdk5 (Bajai, N. P. et al 1999 Prog. Neuropsychopharmacol. Biol. Psychiatr. 23, 833-850)

Stroke

Certain types of neurons, particularly CA1 pyramidal neurons in the hippocampous are vulnerable to ischemic insults such as stroke. It has been shown that ishemic insult in rats leads to accumulation of p25 in CA1 neurons and this is associated with prolonged activation of cdk5. This in turn leads to increased NMDA receptor activity through a specific phosphorylation at Ser 1232 resulting in a massive Ca influx and neuronal death. The effect could be attenuated by inhibiting cdk5 (Wanf, J. et al 2003 Nature Neuroscience 6 (10) 1-9). Inhibiting cdk5 may thus constitute a therapeutic strategy for treating global ischemia due to cardiac arrest.

Parkinson's Disease

Parkinson's disease (PD) is a neurodegenerative disorder characterized by disabeling motor abnormalities including tremor and muscle rigidity. Dysfunction in dopaminergic neurotransmission is considered to be the underlying pathological mechanism of PD. Cdk5 has been shown to act at two levels of the dopamin signalling cascade and furthermore cdk5 inhibitors are capable of increasing both dopamin release and the postsynaptic effects of dopamine (Chergui, K. et al 2004 PNAS 101 2191-2196). In addition, cdk5 inhibition protects nigral neurons from degeneration and improves motor behaviour in the 1-methyl-4-phenyl-1,2,4,6-tetrahydropyridine (MPTP) mouse model of PD (Smith, P. D. et al 2003 PNAS 100 13650-13655). These findings strongly suggest that cdk5 inhibitors might have anti-Parkinsonian actions.

Abuse

Chronic cocaine abuse leads to increase in the expression of the transcription factor ΔFosB and its downstream target cdk5 which seem to be stable compensatory adaptations to accompany chronic cocaine exposure. This upregulation may serve as a homeostatic function to dampen responses to subsequent drug exposure and inhibition of cdk5 has been shown to potentiate the effects of cocaine in vivo (Bibb, J. A. et al 2001 Nature 410 376-380). Cdk5 acts by specifically phosphorylating DARPP-32 (dopamine and cyclic AMP-regualted phosphoprotein, relative molecular mass 32,000) which is involved in regulating dopamine receptor signalling and has thus potential connection to reward-related behaviour connected to abuse of cocaine.

### DISCLOSURE OF THE INVENTION

The object of the present invention is to provide compounds having an inhibiting effect on cyclin-dependent kinase 5 as well as having a good bioavailability.

Accordingly, the present invention provides compounds of formula I:

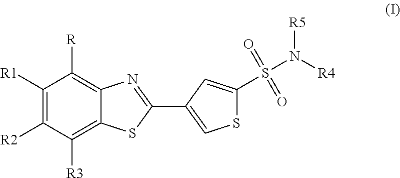

wherein;

R, R¹ and R² are each and independently selected from hydrogen, halo, nitro, CHO, CN, OC₁₋₆alkyl, C(O)C₁₋₄alkyl, C₁₋₆alkyl, C₂₋₆alkenyl, C₂₋₆alkynyl, fluoromethyl, difluoromethyl, trifluoromethyl, fluoromethoxy, difluoromethoxy and trifluoromethoxy;

R³ is selected from hydrogen, halo, C₀₋₆alkylNR⁶R⁷, CO₂R⁸, CONR⁶R⁷, NR⁶(CO)R⁶, O(CO)R⁶, (SO₂)NR⁶R⁷, aryl, or heteroaryl, wherein the aryl and heteroaryl may be substituted by one or more A;

R⁴ and R⁵ are each and independently selected from hydrogen, OH, OC₁₋₆alkyl, C₁₋₆alkyl, C₂₋₆alkenyl, C₂₋₆alkynyl, C₀₋₆alkylC₃₋₆cycloalkyl, CO₂R⁸, C₀₋₆alkylaryl, C₀₋₆alkylheterocycloalkyl, C₁₋₆alkylNR⁶R⁷, and C₀₋₆alkylheteroaryl, wherein any C₁₋₆alkyl, C₂₋₆alkenyl, C₂₋₆alkynyl, C₀₋₆alkylC₃₋₆cycloalkyl, C₀₋₆alkylheterocycloalkyl, C₀₋₆alkylaryl, or C₀₋₆alkylheteroaryl may be substituted by one or more A; or wherein R⁴ and R⁵ may together form a 4-, 5-, 6- or 7-membered heterocyclic ring containing one or more heteroatoms selected from N, O or S, in which said heterocyclic ring may be optionally substituted by A;

R⁶ and R⁷ are each and independently selected from hydrogen, C₁₋₆alkyl, (CO)OR⁸, C₂₋₆alkenyl, C₂₋₆alkynyl, C₀₋₆alkylC₃₋₆cycloalkyl, C₀₋₆alkylaryl and C₀₋₆alkylheteroaryl; or; R⁶ and R⁷ may together form a 4-, 5-, 6- or 7-membered heterocyclic ring containing one or more heteroatoms selected from N, O or S, which heterocyclic ring may be optionally substituted by A;

R⁸ is selected from hydrogen, C₁₋₆alkyl, C₂₋₆alkenyl, C₂₋₆alkynyl, C₀₋₆alkylC₃₋₆cycloalkyl, C₀₋₆alkylaryl and C₀₋₆alkylheteroaryl;

A is halo, nitro, CHO, CN, OR⁶, C₁₋₆alkyl, C₂₋₆alkenyl, C₂₋₆alkynyl, C₀₋₆alkylC₃₋₆cycloalkyl, fluoromethyl, difluoromethyl, trifluoromethyl, fluoromethoxy, difluoromethoxy, trifluoromethoxy, C₀₋₆alkylNR⁶R⁷, OC₁₋₆alkylNR⁶R⁷, C₁₋₆(O)OR⁸, C₁₋₆alkylOR⁶, CONR⁶R⁷, NR⁶(CO)R⁶, O(CO)R⁶, COR⁶, SR⁶, (SO₂)NR⁶R⁷, (SO)NR⁶R⁷, SO₃R⁶, SO₂R⁶ or SOR⁶;

as a free base or a pharmaceutically acceptable salt thereof.

In one aspect of the invention there are provided compounds of formula I, wherein R and R¹ is hydrogen.

In another aspect of the invention there are provided compounds of formula I, wherein R² is selected from hydrogen, halo, nitro, C(O)C₁₋₄alkyl, and C₁₋₆alkyl.

In yet another aspect of the invention there are provided compounds of formula I, wherein R³ is selected from hydrogen, halo and aryl, wherein the aryl may be substituted by one or more A.

In yet another aspect of the invention there are provided compounds of formula I, wherein R⁴ is selected from hydrogen, OH, C₁₋₆alkyl, C₀₋₆alkylaryl and C₀₋₆alkylheteroaryl, wherein any C₁₋₆alkyl, C₀₋₆alkylaryl, or C₀₋₆alkylheteroaryl may be substituted by one or more A.

In yet another aspect of the invention there are provided compounds of formula I, wherein R⁵ is hydrogen.

In yet another aspect of the invention there are provided compounds of formula I, wherein R⁴ and R⁵ together form a 4-, 5-, 6- or 7-membered heterocyclic ring containing one or more heteroatoms selected from N, O or S, in which said heterocyclic ring may be optionally substituted by A.

In yet another aspect of the invention there are provided compounds of formula I, wherein A is selected from halo, OR⁶, C₁₋₆alkyl, and C₁₋₆alkylOR⁶; R⁶ is selected from hydrogen and (CO)OR⁸; and R⁸ is C₁₋₆alkyl.

In yet another aspect of the invention there are provided compounds of formula I, wherein R and R¹ is hydrogen; R² is selected from hydrogen, halo, nitro, C(O)C₁₋₄alkyl, and C₁₋₆alkyl; R³ is selected from hydrogen, halo and aryl, wherein said aryl may be substituted by one or more A; R⁴ is selected from hydrogen, OH, C₁₋₆alkyl, C₀₋₆alkylaryl and C₀₋₆alkylheteroaryl, wherein any C₁₋₆alkyl, C₀₋₆alkylaryl, or C₀₋₆alkylheteroaryl may be substituted by one or more A; R⁵ is hydrogen; and A is selected from halo, OR⁶, C₁₋₆alkyl, and C₁₋₆alkylOR⁶; R⁶ is selected from hydrogen and (CO)OR⁸; and R⁸ is C₁₋₆alkyl.

In yet another aspect of the invention there are provided compounds of formula I, wherein R and R¹ is hydrogen; R² is selected from hydrogen, halo, nitro, C(O)C₁₋₄alkyl, and C₁₋₆alkyl; R³ is selected from hydrogen, halo and aryl, wherein said aryl may be substituted by one or more A; R⁴ and R⁵ together form a 4-, 5-, 6- or 7-membered heterocyclic ring containing one or more heteroatoms selected from N, O or S, in which said heterocyclic ring may be optionally substituted by A, said A being C₁₋₆alkylOR⁶; and R⁶ is hydrogen.

Yet another aspect of the invention relates to compounds selected from:

6-Nitro-2-thiophen-3-yl-benzothiazole;

6-Methyl-2-thiophen-3-yl-benzothiazole;

6-Fluoro-2-thiophen-3-yl-benzothiazole;

6-Chloro-2-thiophen-3-yl-benzothiazole;

1-(2-Thiophen-3-yl-benzothiazol-6-yl)-ethanone;

7-(4-Fluoro-phenyl)-benzothiazole;

7-(4-Fluoro-phenyl)-2-thiophen-3-yl-benzothiazole;

2-Bromo-7-(4-fluoro-phenyl)-benzothiazole;

4-(6-Nitro-benzothiazol-2-yl)-thiophene-2-sulfonic acid;

4-(6-Methyl-benzothiazol-2-yl)-thiophene-2-sulfonic acid;

4-(6-Fluoro-benzothiazol-2-yl)-thiophene-2-sulfonic acid;

4-(6-Chloro-benzothiazol-2-yl)-thiophene-2-sulfonic acid;

4-(6-Acetyl-benzothiazol-2-yl)-thiophene-2-sulfonic acid;

4-[7-(4-Fluoro-phenyl)-benzothiazol-2-yl]-thiophene-2-sulfonic acid;

4-(Benzothiazole-2-yl)-thiophene-2-sulfonyl chloride;

4-(6-Nitro-benzothiazol-2-yl)-thiophene-2-sulfonyl chloride;

4-(6-Methyl-benzothiazol-2-yl)-thiophene-2-sulfonyl chloride;

4-(6-Fluoro-benzothiazol-2-yl)-thiophene-2-sulfonyl chloride;

4-(6-Chloro-benzothiazol-2-yl)-thiophene-2-sulfonyl chloride;

4-[7-(4-Fluoro-phenyl)-benzothiazol-2-yl]-thiophene-2-sulfonyl chloride;

4-(6-Acetyl-benzothiazol-2-yl)-thiophene-2-sulfonyl chloride;

1-(2-Amino-benzothiazol-6-yl)-ethanone;

2-Bromo-6-nitrobenzothiazole;

2-Bromo-6-methylbenzothiazole;

2-Bromo-6-fluorobenzothiazole;

2-Bromo-6-chlorobenzothiazole;

1-(2-Bromo-benzothiazol-6-yl)-ethanone;

2-Bromo-7-chloro-6-fluoro-benzothiazole;

7-Chloro-6-fluoro-2-thiophen-3-yl-benzothiazole; and

7-Bromo-benzothiazole.

Yet another aspect of the invention relates to compounds selected from:

4-(Benzothiazol-2-yl)-thiophene-2-sulfonic acid amide;

4-(6-Nitro-benzothiazol-2-yl)-thiophene-2-sulfonic acid amide;

4-(6-Methyl-benzothiazol-2-yl)-thiophene-2-sulfonic acid amide;

4-(6-Fluoro-benzothiazol-2-yl)-thiophene-2-sulfonic acid amide;

4-(6-Chloro-benzothiazol-2-yl)-thiophene-2-sulfonic acid amide;

4-(6-Acetyl-benzothiazol-2-yl)-thiophene-2-sulfonic acid amide;

4-[7-(4-Fluoro-phenyl)-benzothiazol-2-yl]-thiophene-2-sulfonic acid amide;

4-(7-Chloro-6-fluoro-benzothiazol-2-yl)-thiophene-2-sulfonic acid amide;

4-Benzothiazol-2-yl-thiophene-2-sulfonic acid (2-fluoro-ethyl)-amide;

4-Benzothiazol-2-yl-thiophene-2-sulfonic acid thiazol-2-ylamide;

4-Benzothiazol-2-yl-thiophene-2-sulfonic acid (4,5-dimethyl-thiazol-2-yl)-amide;

4-Benzothiazol-2-yl-thiophene-2-sulfonic acid (3-hydroxy-pyridin-2-yl)-amide;

4-Benzothiazol-2-yl-thiophene-2-sulfonic acid (6-morpholin-4-yl-pyridin-3-yl)-amide;

4-Benzothiazol-2-yl-thiophene-2-sulfonic acid (pyridin-2-ylmethyl)-amide;

[1-(4-Benzothiazol-2-yl-thiophene-2-sulfonyl)-pyrrolidin-2-yl]-methanol;

4-Benzothiazol-2-yl-thiophene-2-sulfonic acid hydroxyamide;

[2-(4-Benzothiazol-2-yl-thiophene-2-sulfonylamino)-thiazol-4-yl]-acetic acid ethyl ester: and

4-Benzothiazol-2-yl-thiophene-2-sulfonic acid (4-methyl-pyridin-2-yl)-amide.

Listed below are definitions of various terms used in the specification and claims to describe the present invention.

In this specification the term “alkyl” includes both straight and branched chain alkyl groups. The term C₁₋₆alkyl having 1 to 6 carbon atoms and may be methyl, ethyl, n-propyl, i-propyl, n-butyl, i-butyl, s-butyl, t-butyl, n-pentyl, i-pentyl, t-pentyl, neo-pentyl, n-hexyl or i-hexyl. The term C₁₋₃alkyl having 1 to 3 carbon atoms and may be methyl, ethyl, n-propyl or i-propyl. The term C₁₋₂alkyl having 1 to 2 carbon atoms and may be methyl or ethyl.

A similar convention applies to other radicals, for example “C₀₋₆alkylaryl” includes 1-phenylethyl and 2-phenylethyl.

In the case where a subscript is the integer 0 (zero) the group to which the subscript refers to indicates that the group may be absent, i.e. there is a direct bond between the groups.

The term “cycloalkyl” refers to an optionally substituted, saturated cyclic hydrocarbon ring system. The term “C₃₋₆cycloalkyl” may be cyclopropyl, cyclobutyl, cyclopentyl or cyclohexyl.

The term “alkenyl” refers to a straight or branched chain alkenyl group. The term C₂₋₆alkenyl having 2 to 6 carbon atoms and one double bond, and may be vinyl, allyl, propenyl, i-propenyl, butenyl, i-butenyl, crotyl, pentenyl, i-pentenyl or hexenyl. The term C₂₋₃alkenyl having 2 to 3 carbon atoms and one or two double bond, and may be vinyl, allyl, propenyl or i-propenyl.

The term “alkynyl” refers to a straight or branched chain alkynyl groups. The term C₂₋₆alkynyl having 2 to 6 carbon atoms and one triple bond, and may be ethynyl, propargyl, butynyl, i-butynyl, pentynyl, i-pentynyl or hexynyl. The term C₂₋₃alkynyl having 2 to 3 carbon atoms and one triple bond, and may be ethynyl or propargyl.

The term “halo” refers to fluoro, chloro, bromo and iodo.

The term “aryl” refers to an optionally substituted monocyclic or bicyclic hydrocarbon ring system containing at least one unsaturated aromatic ring. The “aryl” may be fused with a C₅₋₇cycloalkyl ring to form a bicyclic hydrocarbon ring system. Examples include phenyl, naphthyl, indanyl or tetralinyl.

The term “heteroaryl” as used herein is defined as one or more aromatic rings having 5-14 carbon atoms, including both single rings and polycyclic rings, such as imidazopyridine, in which one or several of the ring carbon atoms is replaced by either oxygen, nitrogen or sulphur, such as furyl, imidazolyl, isoxazolyl, isothiazolyl, oxazolyl, pyrazinyl, pyrazolyl, pyridazinyl, pyridyl, pyrimidyl, pyrrolyl, thiazolyl or thienyl.

The term “heterocycloalkyl” and “4-, 5-, 6-, or 7-membered heterocyclic ring containing one or more heteroatoms selected from N, O or S” may optionally contain a carbonyl function and is preferably a 5, 6 or 7 membered heterocyclic ring and may be imidazolidinyl, imidazolinyl, morpholinyl, piperazinyl, piperidinyl, piperidonyl, pyrazolidinyl, pyrazolinyl, pyrrolidinyl, pyrrolinyl, 1-methyl-1,4-diazepane, tetrahydropyranyl, thiomorpholinyl. In the case where the heterocyclic ring contains a heteroatom selected from S this includes optionally SO and SO₂.

The present invention relates to the use of compounds of formula I as hereinbefore defined as well as to the salts thereof. Salts for use in pharmaceutical compositions will be pharmaceutically acceptable salts, but other salts may be useful in the production of the compounds of formula I.

Both organic and inorganic acids can be employed to form non-toxic pharmaceutically acceptable salts of the compounds of this invention. Pharmaceutically acceptable salts include, but are not limited to hydrochloride, and fumarate. These salts are readily prepared by methods known in the art.

Some compounds of formula I may have chiral centres and/or geometric isomeric centres (E- and Z-isomers), and it is to be understood that the invention encompasses all such optical, diastereoisomeric and geometric isomers.

Pharmaceutical Compositions

According to one aspect of the present invention there is provided a pharmaceutical composition comprising a compound of formula I, as a free base or a pharmaceutically acceptable salt thereof, for use in the prevention and/or treatment of conditions associated with cyclin-dependent kinase 5.

The composition may be in a form suitable for oral administration, for example as a tablet, for parenteral injection as a sterile solution or suspension. In general the above compositions may be prepared in a conventional manner using pharmaceutically carriers or diluents. Suitable daily doses of the compounds of formula I in the treatment of a mammal, including man, are approximately 0.01 to 250 mg/kg bodyweight at peroral administration and about 0.001 to 250 mg/kg bodyweight at parenteral administration. The typical daily dose of the active ingredients varies within a wide range and will depend on various factors such as the relevant indication, the route of administration, the age, weight and sex of the patient and may be determined by a physician.

A compound of formula I, or a pharmaceutically acceptable salt thereof, can be used on its own but will usually be administered in the form of a pharmaceutical composition in which the formula I compound/salt (active ingredient) is in association with a pharmaceutically acceptable diluent or carrier. Dependent on the mode of administration, the pharmaceutical composition may comprise from 0.05 to 99% w (per cent by weight), for example from 0.10 to 50% w, of active ingredient, all percentages by weight being based on total composition.

A diluent or carrier includes water, aqueous polyethylene glycol, magnesium carbonate, magnesium stearate, talc, a sugar (such as lactose), pectin, dextrin, starch, tragacanth, microcrystalline cellulose, methylcellulose, sodium carboxymethyl cellulose or cocoa butter.

A composition of the invention can be in tablet or injectable form. The tablet may additionally comprise a disintegrant and/or may be coated (for example with an enteric coating or coated with a coating agent such as hydroxypropyl methylcellulose).

The invention further provides a process for the preparation of a pharmaceutical composition of the invention which comprises mixing a compound of formula I, or a pharmaceutically acceptable salt thereof, a hereinbefore defined, with a pharmaceutically acceptable diluent or carrier.

An example of a pharmaceutical composition of the invention is an injectable solution containing a compound of the invention, or a a pharmaceutically acceptable salt thereof, as hereinbefore defined, and sterile water, and, if necessary, either sodium hydroxide or hydrochloric acid to bring the pH of the final composition to about pH 5, and optionally a surfactant to aid dissolution.

Liquid solution comprising a compound of formula I, or a salt thereof, dissolved in water.

| Solution | mg/mL |
|---|---|
| Active Compound | 5.0% w/v |
| Pure water | To 100% |

Medical Use

Surprisingly, it has been found that the compounds defined in the present invention, as a free base or a pharmaceutically acceptable salt thereof, are well suited for inhibiting cyclin-dependent kinase 5. Accordingly, the compounds of the present invention are expected to be useful in the prevention and/or treatment of conditions associated with cyclin-dependent kinase 5 activity, i.e. the compounds may be used to produce an inhibitory effect of cyclin-dependent kinase 5 in mammals, including man, in need of such prevention and/or treatment.

Cyclin-dependent kinase 5 is highly expressed in the central and peripheral nervous system and in other tissues. Thus, it is expected that compounds of the invention are well suited for the prevention and/or treatment of conditions associated with cyclin-dependent kinase 5 in the central and peripheral nervous system. In particular, the compounds of the invention are expected to be suitable for prevention and/or treatment of conditions associated with especially, dementia, Alzheimer's Disease, Parkinson's Disease, Frontotemporal dementia Parkinson's Type, Parkinson dementia complex of Guam, HIV dementia, diseases with associated neurofibrillar tangle pathologies, progressive supranuclear palsy, and dementia pugilistica.

Other conditions are selected from the group consisting of amyotrophic lateral sclerosis, corticobasal degeneration, Down syndrome, Huntington's Disease, postencephelatic parkinsonism, progressive supranuclear palsy, Pick's Disease, Niemann-Pick's Disease, stroke, head trauma and other chronic neurodegenerative diseases.

Further conditions are selected from the group of stroke and chronic drug abuse.

One embodiment of the invention relates to the prevention and/or treatment of dementia and Alzheimer's Disease.

The dose required for the therapeutic or preventive treatment of a particular disease will necessarily be varied depending on the host treated, the route of administration and the severity of the illness being treated.

The present invention relates also to the use of a compound of formula I as defined hereinbefore, in the manufacture of a medicament for the prevention and/or treatment of conditions associated with cyclin-dependent kinase 5.

In the context of the present specification, the term “therapy” also includes “prevention” unless there are specific indications to the contrary. The terms “therapeutic” and “therapeutically” should be construed accordingly.

The invention also provides for a method of treatment and/or prevention of conditions associated with cdk-5 comprising administrering to a mammal, including man in need of such treatment and/or prevention a therapeutically effective amount of a compound of formula I, as hereinbefore defined.

Non-Medical Use

In addition to their use in therapeutic medicine, the compounds of formula I as a free base or a pharmaceutically acceptable salt thereof, are also useful as pharmacological tools in the development and standardisation of in vitro and in vivo test systems for the evaluation of the effects of inhibitors of cyclin-dependent kinase 5 related activity in laboratory animals such as cats, dogs, rabbits, monkeys, rats and mice, as part of the search for new therapeutics agents.

Methods of Preparation

Another aspect of the present invention provides a process for preparing a compound of formula I as a free base or a pharmaceutically acceptable salt thereof.

Throughout the following description of such processes it is understood that, where appropriate, suitable protecting groups will be added to, and subsequently removed from, the various reactants and intermediates in a manner that will be readily understood by one skilled in the art of organic synthesis. Conventional procedures for using such protecting groups as well as examples of suitable protecting groups are described, for example, in “Protective Groups in Organic Synthesis” T. W. Green, P. G. M. Wuts, Wiley-Interscience, New York, 1999.

Methods of Preparation of the Intermediates

The process for the preparation of intermediates, wherein R, R¹, R², R³, R⁴, R⁵, R⁶, R⁷ and R⁸ are, unless specified otherwise, defined as in formula I, comprises of:

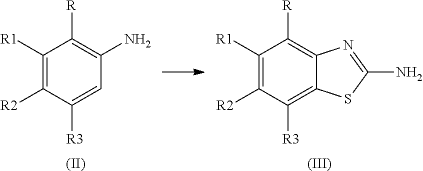

(i) formation of compounds of formula III may be obtained by reacting an appropriate aniline II with bromine and potassium thiocyanate or ammonium thiocyanate. The reaction may be performed in a suitable solvent such as acetic acid at ambient temperature.

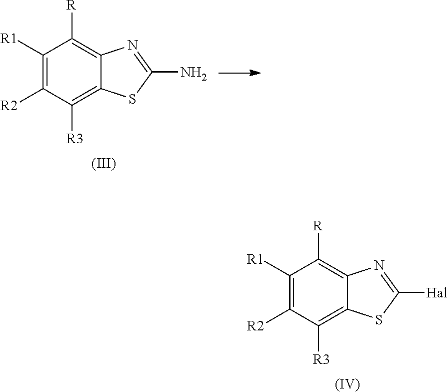

(ii) halo-de-diazoniation of a compound of formula III to obtain a compound of formula IV, may be carried out by formation of the diazonium salt of a compound of formula III and subsequent treatment of the formed diazonium salt with a suitable copper halide or a reagents such as copper and HBr or HCl. The diazonium salts may be obtained using isoamyl nitrite or nitrous acid in a suitable solvent such as acetonitrile-polyethylene glycol.

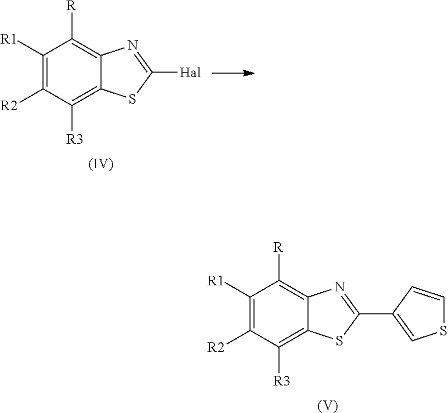

(iii) reaction of a compound of formula IV with 3-thienylboronic acid to obtain a compound of formula V. The reaction may be carried out using a suitable palladium catalyst such as Pd(PPh₃)₄, Pd(dppf)Cl₂ or Pd(OAc)₂ together with a suitable ligand such as P(tert-butyl)₃ or 2-(dicyclohexylphosphino)biphenyl or a nickel catalyst such as nickel on charcoal or Ni(dppe)Cl₂ together with Zn and sodium triphenylphosphinetrimetasulfonate. A suitable base such as an alkyl amine e.g. triethyl amine, or potassium carbonate, sodium carbonate, sodium hydroxide or cesium fluoride may be used in the reaction, which is performed in a temperature range between +20° C. and +160° C. using an oil bath or a microwave oven in a suitable solvent or solvent mixture such as toluene, tetrahydrofuran, dimethoxyethane/water or N,N-dimethylformamide.

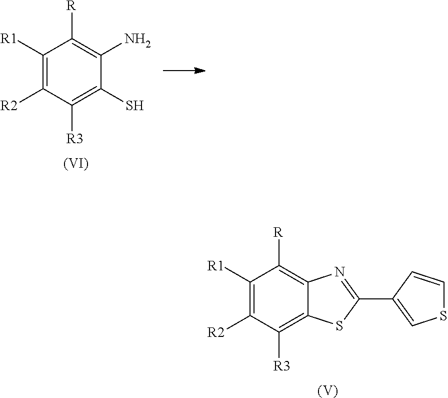

(iv) compounds of formula V can also be obtained by reacting an appropriate anilinothiol VI with thiophene-3-carboxylic acid in a suitable solvent such as ployphosphoric acid or xylene at a reaction temperature between +70° C. and +230° C.

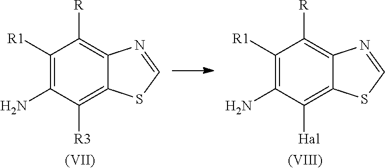

(v) halogenation of a compound of formula VII wherein R3 is hydrogen, to obtain a compound of formula VIII, may be carried out using a suitable halogenating reagent such as iodine, bromine, chlorine, halide salts such as ICl, BrCl or HOCl or other suitable halogenation reagents such as N-bromosuccinimide or phosphorous tribromide. The reaction may be catalysed by metals or acids such as Fe, Cu-salts, acetic acid or sulfuric acid or aided by oxidising agents such as nitric acid, hydrogen peroxide or sulfur trioxide. The reaction may be carried out in a suitable solvent such as water, acetic acid or chloroform at a temperature in the range of −70° C. to +100° C.

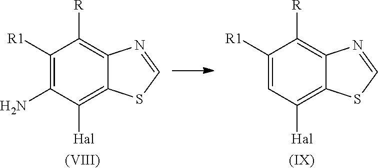

(vi) dediazoniation of a diazonium salt of a compound of formula VIII to obtain a compound of formula IX. Formation of the diazonium salt may be obtained by using isoamyl nitrite or nitrous acid followed by reduction of the diazonium group by a suitable reagent such as hypophosphorous acid, sodium borohydride or ethanol.

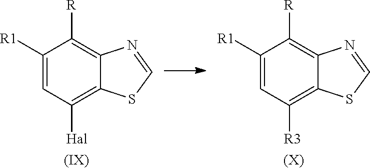

(vii) coupling of a compound of formula IX with an appropriate aryl boronic acid or a boronic ester to give a compound of formula X. The reaction may be carried out using a suitable palladium catalyst such as Pd(PPh₃)₄, Pd(dppf)Cl₂ or Pd(OAc)₂ together with a suitable ligand such as P(tert-butyl)₃ or 2-(dicyclohexylphosphino)biphenyl or a nickel catalyst such as nickel on charcoal or Ni(dppe)Cl₂ together with Zn and sodium triphenylphosphinetrimetasulfonate. A suitable base such as an alkyl amine e.g. triethyl amine, or potassium carbonate, sodium carbonate, sodium hydroxide or cesium fluoride may be used in the reaction, which is performed in the temperature range between +20° C. and +160° C. using an oil bath or in a microwave oven in a suitable solvent or solvent mixture such as toluene, tetrahydrofuran, dimethoxyethane/water or N,N-dimethylformamide;

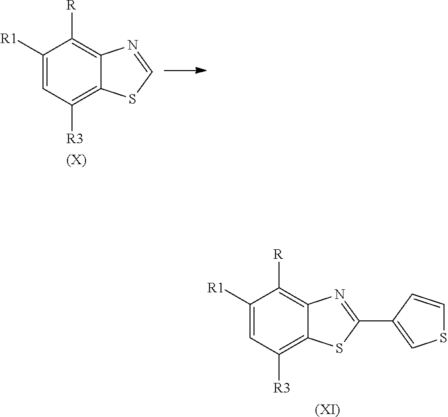

(viii) reaction of a compound of formula X with a suitable base such as a butyllithium and subsequent halogenation with a suitable halogenation reagent such as carbon tetrabromide, followed by coupling with 3-thienylboronic acid to obtain a compound of formula XI. The coupling may be carried out using a suitable palladium catalyst such as Pd(PPh₃)₄, Pd(dppf)Cl₂ or Pd(OAc)₂ together with a suitable ligand such as P(tert-butyl)₃ or 2-(dicyclohexylphosphino)biphenyl or a nickel catalyst such as nickel on charcoal or Ni(dppe)Cl₂ together with Zn and sodium triphenylphosphinetrimetasulfonate. A suitable base such as an alkyl amine e.g. triethyl amine, or potassium carbonate, sodium carbonate, sodium hydroxide or cesium fluoride may be used in the reaction, which is performed in the temperature range between +20° C. and +160° C. using an oil bath or in a microwave oven in a suitable solvent or solvent mixture such as toluene, tetrahydrofuran, s dimethoxyethane/water or N,N-dimethylformamide;

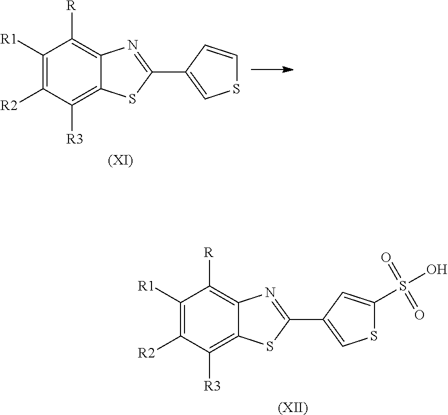

(ix) formation of a sulfonic acid of formula XII may be performed by the treatment of a compound of formula XI with a suitable reagent such as chlorosulfonic acid, sulfuric acid or sulfur trioxide either in a suitable solvent such as chloroform, dichloromethane, 1,2-dichloroethane or carbon tetrachloride or neat at a reaction temperature between −15° C. and +50° C.

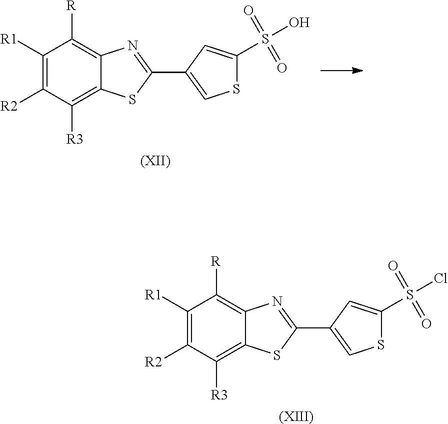

(x) halogenating a compound of formula XII to obtain a compound of formula XIII may be performed by treatment of a compound of formula XII with a halogenation reagents such as PCl₅, PCl₃, thionyl chloride, or oxalyl chloride. The reaction may be performed neat or in a suitable solvent such as POCl₃, dichloromethane, toluene, tetrahydrofuran, dioxane or N,N-dimethylformamide at a temperature range between −20° C. and +140° C.;

Methods of Preparation of End Products

Another object of the invention are processes for the preparation of a compound of general formula I, wherein R, R¹, R², R³, R⁴, R⁵, R⁶, R⁷ and R⁸ are, unless specified otherwise, defined as in formula I, comprising of:

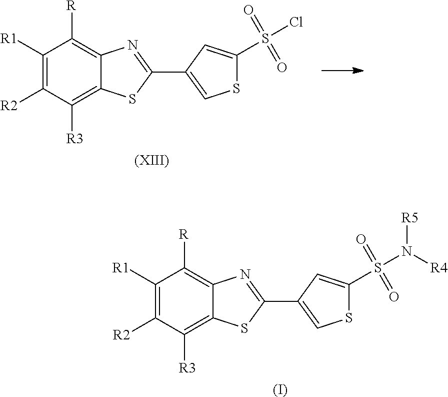

Amidation of a compound of formula XIII to obtain a compound of formula I may be carried out by reacting a compound of formula XIII with a suitable amine. A suitable base such as pyridine, which may also be used as solvent, may be used in the reaction. The reaction may be performed in a suitable solvent such as tetrahydrofuran, dichloromethane, dioxane or N,N-dimethylformamide at a reaction temperature between 0° C. to +100° C. When R⁴ and R⁵ are defined as H, ammonia is used in a suitable solvent such as methanol at ambient temperature.

Consequently, in one aspect there is provided a process for the preparation of a compound of formula I according to claim 1, wherein R, R¹, R², R³, R⁴, R⁵ are, unless specified otherwise, defined as in formula I of claim 1,

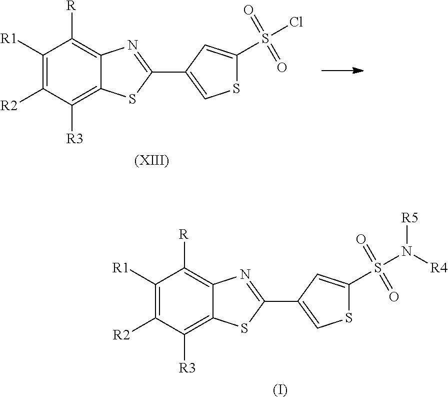

comprising amidating a compound of formula XIII with a suitable amine in the presence of a suitable solvent, to obtain a compound of formula I.

### EXAMPLES

The following examples will now be illustrated by the following non-limiting examples.

Abbreviations

GC-MS gas chromatography mass spectroscopy

HPLC high performance liquid chromatography

LC-MS liquid chromatography mass spectroscopy

NMR nuclear magnetic resonance

δ chemical shift in ppm

br broad

d doublet

m multiplet

q quartet

s singlet

t triplet

General Methods

All starting materials are commercially available or earlier described in the literature. The 1H NMR spectra were recorded on Brucker 400 at 400 MHz or on Bruker Avance operating at 300 MHz. Chemical shifts are given in ppm.

The mass spectra were recorded utilising thermospray (Finnigan MAT SSQ 7000, buffer: 50 nM NH₄OAc in CH₃CN:H₂O; 3:7, electron impact (Finnigan MAT SSQ 710) or electrospray (LC-MS;LC:Waters 2790 or LC-MS, Waters 2690, column XTerra MS C8 2.5 μm 2.1×30 mm, buffer gradient H₂O+0.1% TFA:CH₃CN+0.04% TFA, MS:micromass ZMD ionisation techniques.

GC-MS was performed on an Agilent 6890N GC System using a 5973N Mass Selective Detector.

Column chromatography employed Merck silica gel 60 (40-63 μm).

Purifications were performed on either a semi preparative HPLC with a mass triggered fraction collector, Shimadzu QP 8000, equipped with an XTerra 5 μm C18 100 mm×19 mm column, or on a Waters FractionLynx HPLC with a mass triggered fraction collector, equipped with an Ace C8 5 μm 100 mm×21.2 mm column.

### Example 1

### 6-Nitro-2-thiophen-3-yl-benzothiazole

3-Thiopheneboronic acid (1.3 g, 10.1 mmol) and a saturated aqueous solution of sodium to carbonate (12 mL) was added to a solution of 2-bromo-6-nitrobenzothiazole (2.0 g, 7.72 mmol, described in Lópes-Calahorra et al. Tetrahedron, 2004, 60, 285-289) in toluene/ethanol (29 mL, 9:2) followed by addition of [1,1′-bis(diphenylphosphino)ferrocene]palladium(II) chloride (0.254 g, 0.35 mmol), and the reaction was stirred for 9 h at 80° C. under an atmosphere of nitrogen. Dichloromethane and water were added and the layers separated. The aqueous phase was extracted with dichloromethane (2×15 mL) and the combined organic layers were washed with water and brine, dried over magnesium sulfate, and the solvent was evaporated. Purification of the residue by column chromatography on silica using n-heptane/ethyl acetate (100 to 90:10), as the eluent afforded 0.301 g (15% yield) the title compound: ¹H NMR (CDCl₃, 300 MHz) δ 8.82 (d, 1H), 8.36 (dd, 1H), 8.11 (m, 2H), 7.73 (dd, 1H), 7.49 (m, 1H).

The following Examples 2-7, were synthesized as described for Example 1.

### Example 2

### 6-Methyl-2-thiophen-3-yl-benzothiazole

Starting material: 2-Bromo-6-methylbenzothiazole, yield 20%: ¹H NMR (CDCl₃, 300 MHz) δ 7.98 (dd, 1H), 7.92 (d, 1H), 7.69 (dd, 111), 7.67 (s, 1H), 7.43 (dd, 1H), 7.29 (dd, 1H) and 2.53 (s, 3H).

### Example 3

### 6-Fluoro-2-thiophen-3-yl-benzothiazole

Starting material: 2-Bromo-6-fluorobenzothiazole, yield 33%: ¹H NMR (DMSO-d₆, 300 MHz) δ 8.38 (d, 1H), 8.05 (m, 2H), 7.79 (m, 1H), 7.71 (d, 1H), 7.40 (dt, 1H).

### Example 4

### 6-Chloro-2-thiophen-3-yl-benzothiazole

Starting material: 2-Bromo-6-chlorobenzothiazole, yield 23%: LC-MS (ES) m/z 252 (M⁺+1).

### Example 5

### 1-(2-Thiophen-3-yl-benzothiazol-6-yl)-ethanone

Starting material: 1-(2-Brorno-benzothiazol-6-yl)-ethanone, yield 66%: LC-MS (ES) m/z 260 (M⁺+1).

### Example 6

### 7-(4-Fluoro-phenyl)-benzothiazole

Starting materials: 7-Bromo-benzothiazole and 4-fluorophenylboronic acid, yield 48%: LC-MS (ES) m/z 230 (M⁺+1).

### Example 7

### 7-(4-Fluoro-phenyl)-2-thiophen-3-yl-benzothiazole

Starting material: 2-Bromo-7-(4-fluoro-phenyl)-benzothiazole, yield 69%: LC-MS (ES) m/z 312 (M⁺+1).

### Example 8

### 2-Bromo-7-(4-fluoro-phenyl)-benzothiazole

n-Buthyllithium (0.2 mL, 2.5 M in hexane, 0.5 mmol) was added to a cooled (−78° C.) solution of 7-(4-fluoro-phenyl)-benzothiazole (0.100 g, 0.44 mmol) in dry tetrahydrofuran (10 mL). The resulting mixture was stirred at −78° C. for 1 h and carbon tetrabromide (0.138 g, 0.42 mmol) was added, the cooling was removed and the mixture was allowed to warm up to room temperature. A saturated aqueous solution of ammonium chloride was added and the mixture was diluted with ethyl acetate. The organic layer was dried over magnesium sulfate and the solvent was removed in vacuo. The residue was purified by preparative HPLC to give 45 mg (39% yield) of the title product as a white solid: LC-MS (ES) m/z 308 (M⁺+1).

### Example 9

### 4-(6-Nitro-benzothiazol-2-yl)-thiophene-2-sulfonic acid

Chlorosulfonic acid (1.15 mL) was added dropwise to a cooled (0° C.) suspension of 6-nitro-2-thiophen-3-yl-benzothiazole (0.301 g, 1.15 mmol), in chloroform (0.7 mL), under an atmosphere of nitrogen, and the mixture was allowed to reach ambient temperature during 5 h. The reaction was poured on ice and the precipitated product was filtered, washed with water and diethyl ether, and dried in vacuo to give 0.320 g (90% yield) of the title compound, which was taken to the next step without further purification.

The following Examples 10-14 were synthesized as described for Example 9:

### Example 10

### 4-(6-Methyl-benzothiazol-2-yl)-thiophene-2-sulfonic acid

Starting material: 6-Methyl-2-thiophen-3-yl-benzothiazole, yield 81%.

### Example 11

### 4-(6-Fluoro-benzothiazol-2-yl)-thiophene-2-sulfonic acid

Starting material: 6-Fluoro-2-thiophen-3-yl-benzothiazole, yield 87%.

### Example 12

### 4-(6-Chloro-benzothiazol-2-yl)-thiophene-2-sulfonic acid

Starting materials: 6-Chloro-2-thiophen-3-yl-benzothiazole, yield 38%: LC-MS (ES) m/z 332 (M⁺+1).

### Example 13

### 4-(6-Acetyl-benzothiazol-2-yl)-thiophene-2-sulfonic acid

Starting material: 1-(2-Thiophen-3-yl-benzothiazol-6-yl)-ethanone, yield 96%: LC-MS (ES) m/z 340 (M⁺+1).

### Example 14

### 4-[7-(4-Fluoro-phenyl)-benzothiazol-2-yl]-thiophene-2-sulfonic acid

Starting material: 7-(4-Fluoro-phenyl)-2-thiophen-3-yl-benzothiazole, yield 63%: LC-MS (ES) m/z 392 (M⁺+1).

### Example 15

### 4-(Benzothiazol-2-yl)-thiophene-2-sulfonyl chloride

Phosphorous pentachloride (0.581 g, 2.79 mmol) was added to a suspension of 4-(benzothiazol-2-yl)-thiophene-2-sulfonic acid (0.554 g, 1.86 mmol, described in Vattoly, J. M. et al. Tetrahedron Lett. 2003, 44, 8535-8537) in phosphorous oxychloride (3.5 mL). The mixture was heated at 100° C. during 2 h, under an atmosphere of nitrogen. The reaction mixture was concentrated, diluted with ethyl acetate and ice water, and the phases were separated. The organic phase was dried over magnesium sulfate and the solvent was evaporated to give 0.533 g (91% yield) of the title compound as a white solid, which was taken to the next step without further purification.

The following Examples 16-20 were synthesized as described for Example 15:

### Example 16

### 4-(6-Nitro-benzothiazol-2-yl)-thiophene-2-sulfonyl chloride

Starting material: 4-(6-Nitro-benzothiazol-2-yl)-thiophene-2-sulfonic acid, yield 96%.

### Example 17

### 4-(6-Methyl-benzothiazol-2-yl)-thiophene-2-sulfonyl chloride

Starting material: 4-(6-Methyl-benzothiazol-2-yl)-thiophene-2-sulfonic acid, yield 100%.

### Example 18

### 4-(6-Fluoro-benzothiazol-2-yl)-thiophene-2-sulfonyl chloride

Starting material: 4-(6-Fluoro-benzothiazol-2-yl)-thiophene-2-sulfonic acid, yield 33%.

### Example 19

### 4-(6-Chloro-benzothiazol-2-yl)-thiophene-2-sulfonyl chloride

Starting material: 4-(6-Chloro-benzothiazol-2-yl)-thiophene-2-sulfonic acid, yield 81%.

### Example 20

### 4-[7-(4-Fluoro-phenyl)-benzothiazol-2-yl]-thiophene-2-sulfonyl chloride

Starting material: 4-[7-(4-Fluoro-phenyl)-benzothiazol-2-yl]-thiophene-2-sulfonic acid, the product was not isolated.

### Example 21

### 4-(6-Acetyl-benzothiazol-2-yl)-thiophene-2-sulfonyl chloride

To a suspension of 4-(6-acetyl-benzothiazol-2-yl)-thiophene-2-sulfonic acid (0.10 g, 0.30 mmol) in phosphorous oxychloride (0.8 mL, 8.8 mmol) was added phosphorous pentachloride (0.1 g, 0.5 mmol). The reaction mixture was stirred at ambient temperature overnight and the solvent was evaporated in vacuo to give 0.070 g (66% yield) of the title compound as a crude, which was taken to the next step without further purification.

### Example 22

### 1-(2-Amino-benzothiazol-6-yl)-ethanone

A solution of bromine (3.79 mL, 74.0 mmol) in acetic acid (50 mL) was added dropwise to a mixture of 4-acetylanilin (10 g, 74.0 mmol), sodium thiocyanate (9.0 g, 111 mmol) and acetic acid (250 mL) at 10° C. over 3 h. The resulting mixture was heated for 2 h at 50° C. After the reaction was cooled to ambient temperature the solid formed was filtered off, suspended in warm water, and basified with sodium hydroxide (pastilles) to pH 9-10. The product was removed by filtration, washed with water, and dried over phosphorous pentoxide in vacuo to afford 5.86 g (41% yield) of the title product as a yellow solid: LC-MS (ES) m/z 193 (M⁺+1).

### Example 23

### 2-Bromo-6-nitrobenzothiazole

A solution of 2-amino-6-nitrobenzthiazole (2.2 g, 11.2 mmol) in acetonitrile (169 mL) and polyethylene glycol (5.5 g) was added dropwise to a mixture of dry copper(II) bromide (6.05 g, 27 mmol) and isoamyl nitrite (2.2 mL, 16.8 mmol) in acetonitrile (55 mL) and polyethylene glycol (5.5 g) under an atmosphere of nitrogen. The mixture was ultra-sonicated and stirred at 50° C. for 3 h. The mixture was cooled (0° C.), poured into hydrogen bromide (10%, 500 mL), and extracted with diethyl ether (2×200 mL). The combined organic phases were washed with hydrogen bromide (10%), neutralized with a saturated aqueous solution of sodium hydrogencarbonate and brine, dried over magnesium sulphate and the solvent was evaporated to give 2.08 g (71% yield) of the title compound as a yellow solid: ¹H NMR (DMSO-d₆, 300 MHz) δ 9.20 (d, 1H), 8.36 (dd, 1H), 8.20 (d, 1H).

The following Examples 24-27 were synthesized as described for Example 23:

### Example 24

### 2-Bromo-6-methylbenzothiazole

Starting material: 2-Amino-6-methylbenzthiazole, yield 91%: ¹H NMR (DMSO-d₆, 300 MHz) δ 7.88 (m, 2H), 7.35 (dd, 1H), 2.43 (s, 3H).

### Example 25

### 2-Bromo-6-fluorobenzothiazole

Starting material: 2-Amino-6-fluorobenzothiazole, yield 92%: ¹H NMR (DMSO-d₆, 300 MHz) δ 8.04 (m, 2H), 7.43 (m, 1H).

### Example 26

### 2-Bromo-6-chlorobenzothiazole

Starting material: 2-Amino-6-chlorobenzothiazole, yield 89%: LC-MS (ES) m/z 250 (M⁺+1).

### Example 27

### 1-(2-Bromo-benzothiazol-6-yl)-ethanone

Starting material: 1-(2-Amino-benzothiazol-6-yl)-ethanone, yield 75%: LC-MS (ES) m/z 256 (M⁺+1).

### Example 28

### 2-Bromo-7-chloro-6-fluoro-benzothiazole, 2-bromo-5-chloro-6-fluoro-benzothiazole

Copper(II) bromide (2.75 g, 12.3 mmol) was dried at 110° C. in vacuo for 1 h. After the flask reached ambient temperature, acetonitrile (90 mL) and polyethylene glycol 200 (3 g) were added followed by isoamyl nitrite (1.05 mL, 7.70 mmol). To this mixture a solution of the isomeric mixture of 7-chloro-6-fluoro-benzothiazol-2-ylamine and 5-chloro-6-fluoro-benzothiazol-2-ylamine (1.04 g, 3:7 ratio, described in Cecchetti, V. J. Med. Chem. 1987, 30, 467-473) in acetonitrile (30 mL) and polyethylene glycol 200 (3 g) was added dropwise over 20 min. The reaction mixture was stirred at 50° C. for 4 h under an atmosphere of nitrogen. After being cooled to ambient temperature, the reaction mixture was poured into cooled (0° C.) 10% hydrogen bromide (400 mL) and extracted with diethyl ether (3×). The combined organic phases were washed with 10% hydrogen bromide, a saturated aqueous solution of sodium hydrogencarbonate and brine, dried over magnesium sulfate, and concentrated in vacuo. The crude material was suspended in n-heptane and filtered. The solid collected was pure undesired isomer (0.738 g, 54% yield). The filtrate was concentrated and dried in vacuo to give 0.481 g (35% yield) of an isomeric mixture containing 70% of the desired regioisomer (2-bromo-7-chloro-6-fluoro-benzothiazole) and 30% of the undesired one according to GC. This mixture was taken to the next step without separation: GC-MS (EI) m/z 267 (M⁺).

### Example 29

### 7-Chloro-6-fluoro-2-thiophen-3-yl-benzothiazole

Thiophene-3-boronic acid (0.255 g, 1.97 mmol), [1,1′-bis(diphenylphosphino)ferrocene]palladium(II) chloride (0.066 g, 0.09 mmol) and a saturated aqueous solution of sodium hydrogencarbonate (5 mL) was added to a solution of the isomeric mixture of 2-bromo-7-chloro-6-fluoro-benzothiazole and 2-bromo-5-chloro-6-fluoro-benzothiazole in toluene/ethanol (10:1) and the reaction was stirred under an atmosphere of nitrogen at 80° C. for 6.5 h. Not completed according to GC-MS. The reaction mixture was left for 18 h at ambient temperature, then additional thiophene-3-boronic acid (0.070 g, 0.541 mmol) and [1,1′-bis(diphenylphosphino)ferrocene]palladium(II) chloride (0.066 g, 0.09 mmol) were added and the reaction stirred under an atmosphere of nitrogen at 80° C. for 5 h. Dichloromethane and water were added, the layers were separated, and the aqueous phase was extracted with dichloromethane (3×). The combined organic phases were washed with water and brine, dried over magnesium sulfate, and concentrated in vacuo. The crude material was purified by column chromatography on silica using n-heptane/ethyl acetate (95:5) as the eluent to obtain 0.196 g (40% yield) of the desired regioisomer (7-chloro-6-fluoro-2-thiophen-3-yl-benzothiazole) as a white solid: ¹H NMR (CDCl₃, 400 MHz) δ 8.03 (m, 1H), 7.89 (dd, 1H), 7.68 (d, 1H), 7.46 (m, 1H), 7.31 (t, 1H); GC-MS (EI) m/z 269 (M⁺).

### Example 30

### 7-Bromo-benzothiazole

Sodium nitrite (0.229 g, 3.3 mmol) was added to a solution of 7-bromo-benzothiazol-6-ylamine (0.380 g, 1.66 mmol, described in WO 97/31636) in sulfuric acid (10 mL) and the resulting mixture was stirred at room temperature for 20 min. Hypophosphorous acid (10 mL) was added and the mixture was heated at 50° C. overnight. The pH was adjusted to 9-10 by addition of sodium carbonate, and the crude product was removed by filtration and washed with water. Purification by prep-HPLC gave 0.265 g (75% yield) of the title compound as a white solid: LC-MS (EI) m/z 213 (M⁺+1).

### Example 31

### 4-(Benzothiazol-2-yl)-thiophene-2-sulfonic acid amide

Ammonia in methanol (1.2 mL, 7 M) was added to the crude 4-(benzothiazol-2-yl)-thiophene-2-sulfonyl chloride (0.060 g, 0.21 mmol) and the resulting mixture was stirred overnight at ambient temperature. The solvent was evaporated and the residue purified by prep-HPLC to give 0.030 g (48% yield) of the title compound as a white solid: ¹H NMR (DMSO-d₆, 400 MHz) δ 8.54 (d, 1H), 8.12 (d, 1H), 8.08 (d, 1H), 7.97 (d, 1H), 7.82 (br s, 2H), 7.50 (t, 1H), 7.42 (t, 1H).

The following Examples 32-37 were synthesized as described for Example 31:

### Example 32

### 4-(6-Nitro-benzothiazol-2-yl)-thiophene-2-sulfonic acid amide

Starting material: 4-(6-Nitro-benzothiazol-2-yl)-thiophene-2-sulfonyl chloride, yield 10%: ¹H NMR (DMSO-d₆, 300 MHz) δ 9.25 (d, 1H), 8.77 (d, 1H), 8.40 (m, 2H), 8.22 (d, 1H), 8.15 (d, 1H), 7.91 (br s, 2H).

### Example 33

### 4-(6-Methyl-benzothiazol-2-yl)-thiophene-2-sulfonic acid amide

Starting material: 4-(6-Methyl-benzothiazol-2-yl)-thiophene-2-sulfonyl chloride, yield 25%: ¹H NMR (DMSO-d₆, 300 MHz) δ 8.55 (d, 1H), 8.08 (d, 1H), 7.93 (m, 2H), 7.38 (d, 1H), 2.44 (s, 3H).

### Example 34

### 4-(6-Fluoro-benzothiazol-2-yl)-thiophene-2-sulfonic acid amide

Starting material: 4-(6-Fluoro-benzothiazol-2-yl)-thiophene-2-sulfonyl chloride, yield 11%: ¹H NMR (DMSO-d₆, 300 MHz) δ 8.60 (d, 1H), 8.03-8.12 (m, 2H) and 7.23-7.59 (m, 4H).

### Example 35

### 4-(6-Chloro-benzothiazol-2-yl)-thiophene-2-sulfonic acid amide

Starting material: 4-(6-Chloro-benzothiazol-2-yl)-thiophene-2-sulfonyl chloride, yield 63%: ¹H NMR (DMSO-d₆, 400 MHz) δ 8.62 (s, 1H), 8.33 (d, 1H), 8.10 (s, 1H), 8.04 (d, 1H), 7.86 (br s, 2H), 7.58 (dd, 1H); MS (TSP) m/z 331 (M⁺+1).

### Example 36

### 4-(6-Acetyl-benzothiazol-2-yl)-thiophene-2-sulfonic acid amide

Starting material: 4-(6-Acetyl-benzothiazol-2-yl)-thiophene-2-sulfonyl chloride, yield 6%: ¹H NMR (DMSO-d₆, 400 MHz) δ 8.86 (s, 1H), 8.70 (1H), 8.11 (m, 3H), 7.84 (br s 2H), 3.30 (s, 3H); LC-MS (ES) m/z 339 (M⁺+1).

### Example 37

### 4-[7-(4-Fluoro-phenyl)-benzothiazol-2-yl]-thiophene-2-sulfonic acid amide

Starting material: 4-[7-(4-Fluoro-phenyl)-benzothiazol-2-yl]-thiophene-2-sulfonyl chloride, yield 63%: ¹H NMR (DMSO-d₆, 400 MHz) δ 8.63 (d, 1H), 8.14 (d, 1H), 8.10 (dd, 1H), 7.89 (br s, 2H), 7.81 (m, 2H), 7.69 (t, 1H), 7.58 (dd, 1H), 7.42 (t, 2H); LC-MS (ES) m/z 391 (M⁺+1).

### Example 38

### 4-(7-Chloro-6-fluoro-benzothiazol-2-yl)-thiophene-2-sulfonic acid amide

Chlorosulfonic acid (2.0 mL) was added dropwise to a cooled (0° C.) suspension of 7-chloro-6-fluoro-2-thiophen-3-yl-benzothiazole (0.192 g, 0.712 mmol) in chloroform (0.70 mL), under an atmosphere of nitrogen. The mixture was allowed to reach ambient temperature and stirred for 5.5 h. The reaction was poured on ice, the precipitated product was filtered off, washed with water and diethyl ether, and dried over phosphorous pentoxide to afford 0.225 g of the desired sulfonic acid and some sulfonyl chloride. This material was suspended in phosphorous oxychloride (2.0 mL), phosphorous pentachloride (0.214 g,) was added, and the mixture was heated at 100° C. during 2 h under an atmosphere of nitrogen. The reaction mixture was concentrated and dried in vacuo. To the crude mixture was added ammonia (7 M in methanol, 2.5 mL) dropwise and the reaction was stirred for 19 h. The reaction mixture was concentrated in vacuo and purified by prep-HPLC to yield 0.042 g (17% yield over 3 steps) of the title compound as a white solid: ¹H NMR (DMSO-d₆, 400 MHz) δ 8.70 (d, 1H), 8.10 (m, 2H), 7.90 (br s, 2H), 7.67 (t, 1H); LC-MS (ES) m/z 346.9 (M⁻−1).

### Example 39

### 4-Benzothiazol-2-yl-thiophene-2-sulfonic acid(2-fluoro-ethyl)-amide

2-Fluoroethyl amine hydrochloride (0.039 g, 0.35 mmol) was added to a solution of 4-(benzothiazol-2-yl)-thiophene-2-sulfonyl chloride (0.092 g, 0.29 mmol) in pyridine (2 mL) and the resulting mixture was stirred at 50° C. for 20 h under an atmosphere of nitrogen. After the mixture was cooled down, water and dichloromethane were added and the layers were separated. The aqueous phase was extracted with dichloromethane (3×) and the combined organic phases were washed with water, a saturated aqueous solution of sodium hydrogencarbonate and brine, dried over magnesium sulfate, and evaporated to give, after purification by prep-HPLC 0.030 g (30% yield) of the title compound: ¹H NMR (DMSO-d₆, 300 MHz) δ 8.67 (d, 1H), 8.16 (d, 1H), 8.12 (d, 1H), 8.06 (d, 1H), 7.57, (m, 1H), 7.48 (m, 1H), 4.53 (t, 1H), 4.37 (t, 2H), 3.28 (t, 1H), 3.19 (t, 1H).

### Examples 40-43

General Procedure MPS:

A mixture of 4-(benzothiazol-2-yl)-thiophene-2-sulfonyl chloride (0.5 mmol) and an appropriate aminoheterocycle (2 eq.) were dissolved in pyridine (1 mL), and the resulting mixture was heated at 100° C. overnight. The solvent was evaporated and the residue was purified by column chromatography on silica using ethyl acetate/dichloromethane (0 to 100% ethyl acetate) as the eluent.

| Ex | Heterocycle | Compound | Yield | MS (ESI) |
|---|---|---|---|---|
| 40 | 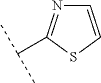 | 4-Benzothiazol-2-yl-thiophene-2- sulfonic acid thiazol-2-ylamide | 30% | 380.0 |
| 41 | 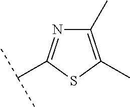 | 4-Benzothiazol-2-yl-thiophene-2- sulfonic acid (4,5-dimethyl- thiazol-2-yl)-amide | 13% | 408.2 |
| 42 | 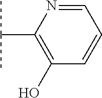 | 4-Benzothiazol-2-yl-thiophene-2- sulfonic acid (3-hydroxy-pyridin- 2-yl)-amide | 40% | 390.0 |
| 43 | 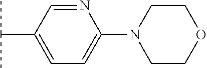 | 4-Benzothiazol-2-yl-thiophene-2- sulfonic acid (6-morpholin-4-yl- pyridin-3-yl)-amide | 58% | 459.1 |

### Examples 44-46

General Procedure MPS:

Using the same procedure as described for Examples 40-43, but heating at 50° C.

| Ex | Heterocycle | Compound | Yield | MS (ES) |
|---|---|---|---|---|
| 44 | 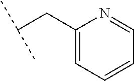 | 4-Benzothiazol-2-yl- thiophene- 2-sulfonic acid (pyridin- 2-ylmethyl)-amide | 41% | 388.1 |
| 45 | 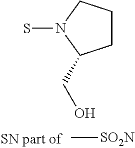 | [1-(4-Benzothiazol-2-yl- thiophene-2-sulfonyl)- pyrrolidin-2-yl]- methanol | 11% | 381.1 |
| 46 | —OH | 4-Benzothiazol-2-yl- thiophene-2-sulfonic acid hydroxyamide | 20% | 313.0 |

### Example 47

### [2-(4-Benzothiazol-2-yl-thiophene-2-sulfonylamino)-thiazol-4-yl]-acetic acid ethyl ester

Pyridine (0.75 mL) was added to a mixture of 4-(benzothiazol-2-yl)-thiophene-2-sulfonyl chloride (0.3 mmol) and (2-amino-thiazol-4-yl)-acetic acid ethyl ester (2 eq.), and the resulting mixture was heated at 50° C. overnight. The solvent was evaporated and the residue was purified by column chromatography on silica using ethyl acetate/dichloromethane (0 to 60% ethyl acetate) as the eluent to afford 0.043 g (30% yield) of the title compound as a solid: ¹H NMR (DMSO-d₆, 400 MHz): δ 13.06 (br s, 1H), 8.60 (d, 1H), 8.17 (d, 1H), 8.07 (d, 1H), 8.04 (d, 1H), 7.57 (dd, 1H), 7.49 (dd, 1H), 6.75 (s, 1H), 4.11 (q, 2H), 3.68 (s, 2H), 1.19 (t, 3H); MS (ES) m/z 465.0 (M⁺+1).

### Example 48

### 4-Benzothiazol-2-yl-thiophene-2-sulfonic acid (4-methyl-pyridin-2-yl)-amide

Pyridine (0.75mL) was added to a mixture of 4-(benzothiazol-2-yl)-thiophene-2-sulfonyl chloride (0.3 mmol) and 4-methyl-pyridin-2-ylamine (2 eq.), and the mixture was heated at 100° C. overnight. The solvent was evaporated and the residue was purified by column chromatography on silica using ethyl acetate/dichloromethane (0 to 60% ethyl acetate) as the eluent, to give 0.041 g (40% yield) of the title compound: ¹H NMR (DMSO-d₆, 400 MHz): δ 13.27 (br s, 1H), 8.53 (s, 1H), 8.17 (d, 1H), 8.06 (d, 1H), 8.04 (s, 1H), 7.87 (d, 1H), 7.57 (dd, 1H), 7.49 (dd, 1H), 7.22 (br s, 1H), 6.75 (d, 1H), 2.33 (s, 3H); MS (ES) m/z 387.9 (M⁺+1).

Pharmacology

Determination of Inhibition of the Cdk5/p25 Protein Kinase in a Scintillation Proximity Assay.

Cdk5/p25 Scintillation Proximity Assay.

The assay experiments were carried out in duplicate with 10 different concentrations of the inhibitors in clear-bottom 384-well microtiter plates (Corning, US, Item No 3706). Recombinant human Cdk5/p25 (Cyclin-dependent kinase 5) (AstraZeneca Biotech Laboratory, Södertälje, Sweden) was added at a final concentration of 3.3 nM in an assay buffer containing 23.3 mM hydroxyethylpiperazineethanesulfonic acid (HEPES), pH 7.35, 0.2 mM ethylenedinitriotetraacetic acid (EDTA), 12.5 mM KCl, 10 mM β-glycerophosphate, 0.02% β-mercaptoethanol, 0.007% Brij 35, 0.8% glycerol and 0.05% bovine serum albumin. After incubation for 15 minutes the reaction was initiated by the addition of 2.95 μM, final concentration, of a biotinylated peptide substrate, Biotin-Ala-Lys-Lys-Pro-Lys-Thr-Pro-Lys-Lys-Ala-Lys-Lys-Leu-OH (Bachem, Switzerland), 0.07 μCi [γ³³P]ATP (Amersham, UK), 2 μM unlabelled ATP and 10 mM MgCl₂ in an final assay volume of 21 μl. After incubation for 40 minutes at room temperature, each reaction was terminated by the addition of 30 μl stop solution containing 24 mM EDTA, 2.2 mM ATP and 0.225 mg streptavidine coated scintillation proximity assay (SPA) beads (Amersham, UK). The microtiter plates were centrifuged for 2 minutes at 200 g and the radioactivity was determined in a liquid scintillation counter (1450 MicroBeta Trilux, Wallac, Finland). The inhibition curves were analysed by non-linear regression using XL-fit. The Kₘ value of ATP for Cdk5/p25, used to calculate the inhibition constants (Kᵢ) of the various compounds, was 10 μM.

Results

Typical Ki values for the compounds of the present invention are in the range of about 275 nM to 10000 nM.

1. A compound according to formula I: 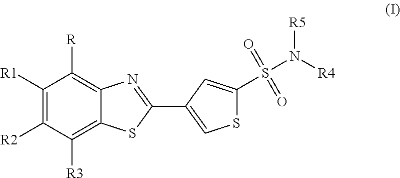
wherein;
R, R¹ and R² are each and independently selected from hydrogen, halo, nitro, CHO, CN, OC₁₋₆alkyl, C(O)C₁₋₄alkyl, C₁₋₆alkyl, C₂₋₆alkenyl, C₂₋₆alkynyl, fluoromethyl, difluoromethyl, trifluoromethyl, fluoromethoxy, difluoromethoxy and trifluoromethoxy;
R³ is selected from hydrogen, halo, C₀₋₆alkylNR⁶R⁷, CO₂R⁸, CONR⁶R⁷, NR⁶(CO)R⁶, O(CO)R⁶, (SO₂)NR⁶R⁷, aryl, or heteroaryl, wherein the aryl and heteroaryl may be substituted by one or more A;
R⁴ and R⁵ are each and independently selected from hydrogen, OH, OC₁₋₆alkyl, C₁₋₆alkyl, C₂₋₆alkenyl, C₂₋₆alkynyl, C₀₋₆alkylC₃₋₆cycloalkyl, CO₂R⁸, C₀₋₆alkylaryl, C₀₋₆alkylheterocycloalkyl, C₁₋₆alkylNR⁶R⁷, and C₀₋₆alkylheteroaryl, wherein any C₁₋₆alkyl, C₂₋₆alkenyl, C₂₋₆alkynyl, C₀₋₆alkylC₃₋₆cycloalkyl, C₀₋₆alkylheterocycloalkyl, C₀₋₆alkylaryl, or C₀₋₆alkylheteroaryl may be substituted by one or more A; or wherein R⁴ and R⁵ may together form a 4-, 5-, 6- or 7-membered heterocyclic ring containing one or more heteroatoms selected from N, O or S, in which said heterocyclic ring may be optionally substituted by A;
R⁶ and R⁷ are each and independently selected from hydrogen, C₁₋₆alkyl, (CO)OR⁸, C₂₋₆alkenyl, C₂₋₆alkynyl, C₀₋₆alkylC₃₋₆cycloalkyl, C₀₋₆alkylaryl and C₀₋₆alkylheteroaryl; or; R⁶ and R⁷ may together form a 4-, 5-, 6- or 7-membered heterocyclic ring containing one or more heteroatoms selected from N, O or S, which heterocyclic ring may be optionally substituted by A;
R⁸ is selected from hydrogen, C₁₋₆alkyl, C₂₋₆alkenyl, C₂₋₆alkynyl, C₀₋₆alkylC₃₋₆cycloalkyl, C₀₋₆alkylaryl and C₀₋₆alkylheteroaryl;
A is halo, nitro, CHO, CN, OR⁶, C₁₋₆alkyl, C₂₋₆alkenyl, C₂₋₆alkynyl, C₀₋₆alkylC₃₋₆cycloalkyl, fluoromethyl, difluoromethyl, trifluoromethyl, fluoromethoxy, difluoromethoxy, trifluoromethoxy, C₀₋₆alkylNR⁶R⁷, OC₁₋₆alkylNR⁶R⁷, C₁₋₆(O)OR⁸, C₁₋₆alkylOR⁶, CONR⁶R⁷, NR⁶(CO)R⁶, O(CO)R⁶, COR⁶, SR⁶, (SO₂)NR⁶R⁷, (SO)NR⁶R⁷, SO₃R⁶, SO₂R⁶ or SOR⁶;
as a free base or a pharmaceutically acceptable salt thereof.

2. A compound according to claim 1, wherein R and R¹ is hydrogen.

3. A compound according to claim 1 or 2, wherein R² is selected from hydrogen, halo, nitro, C(O)C₁₋₄alkyl, and C₁₋₆alkyl.

4. A compound according to any one of claims 1 to 3, wherein R³ is selected from hydrogen, halo and aryl, wherein the aryl may be substituted by one or more A.

5. A compound according to any one of claims 1 to 4, wherein R⁴ is selected from hydrogen, OH, C₁₋₆alkyl, C₀₋₆alkylaryl and C₀₋₆alkylheteroaryl, wherein any C₁₋₆alkyl, C₀₋₆alkylaryl, or C₀₋₆alkylheteroaryl may be substituted by one or more A.

6. A compound according to any one of claims 1 to 5, wherein R⁵ is hydrogen.

7. A compound according to any one of claims 1 to 4, wherein R⁴ and R⁵ together form a 4-, 5-, 6- or 7-membered heterocyclic ring containing one or more heteroatoms selected from N, O or S, in which said heterocyclic ring may be optionally substituted by A.

8. A compound according to any one of claims 1 to 7, wherein A is selected from halo, OR⁶, C₁₋₆alkyl, and C₁₋₆alkylOR⁶; R⁶ is selected from hydrogen and (CO)OR⁸; and R⁸ is C₁₋₆alkyl.

9. A compound according to any one of claims 1 to 8, wherein R and R¹ is hydrogen; R² is selected from hydrogen, halo, nitro, C(O)C₁₋₄alkyl, and C₁₋₆alkyl; R³ is selected from hydrogen, halo and aryl, wherein said aryl may be substituted by one or more A; R⁴ is selected from hydrogen, OH, C₁₋₆alkyl, C₀₋₆alkylaryl and C₀₋₆alkylheteroaryl, wherein any C₁₋₆alkyl, C₀₋₆alkylaryl, or C₀₋₆alkylheteroaryl may be substituted by one or more A; R⁵ is hydrogen; and A is selected from halo, OR⁶, C₁₋₆alkyl, and C₁₋₆alkylOR⁶; R⁶ is selected from hydrogen and (CO)OR⁸; and R⁸ is C₁₋₆alkyl.

10. A compound according to claim 9, wherein R⁴ and R⁵ together form a 4-, 5-, 6- or 7-membered heterocyclic ring containing one or more heteroatoms selected from N, O or S, in which said heterocyclic ring may be optionally substituted by A, said A being C₁₋₆alkylOR⁶; and R⁶ is hydrogen.

11. A compound which is:
6-Nitro-2-thiophen-3-yl-benzothiazole;
6-Methyl-2-thiophen-3-yl-benzothiazole;
6-Fluoro-2-thiophen-3-yl-benzothiazole;
6-Chloro-2-thiophen-3-yl-benzothiazole;
1-(2-Thiophen-3-yl-benzothiazol-6-yl)-ethanone;
7-(4-Fluoro-phenyl)-benzothiazole;
7-(4-Fluoro-phenyl)-2-thiophen-3-yl-benzothiazole;
2-Bromo-7-(4-fluoro-phenyl)-benzothiazole;
4-(6-Nitro-benzothiazol-2-yl)-thiophene-2-sulfonic acid;
4-(6-Methyl-benzothiazol-2-yl)-thiophene-2-sulfonic acid;
4-(6-Fluoro-benzothiazol-2-yl)-thiophene-2-sulfonic acid;
4-(6-Chloro-benzothiazol-2-yl)-thiophene-2-sulfonic acid;
4-(6-Acetyl-benzothiazol-2-yl)-thiophene-2-sulfonic acid;
4-[7-(4-Fluoro-phenyl)-benzothiazol-2-yl]-thiophene-2-sulfonic acid;
4-(Benzothiazole-2-yl)-thiophene-2-sulfonyl chloride;
4-(6-Nitro-benzothiazol-2-yl)-thiophene-2-sulfonyl chloride;
4-(6-Methyl-benzothiazol-2-yl)-thiophene-2-sulfonyl chloride;
4-(6-Fluoro-benzothiazol-2-yl)-thiophene-2-sulfonyl chloride;
4-(6-Chloro-benzothiazol-2-yl)-thiophene-2-sulfonyl chloride;
4-[7-(4-Fluoro-phenyl)-benzothiazol-2-yl]-thiophene-2-sulfonyl chloride;
4-(6-Acetyl-benzothiazol-2-yl)-thiophene-2-sulfonyl chloride;
1-(2-Amino-benzothiazol-6-yl)-ethanone;
2-Bromo-6-nitrobenzothiazole;
2-Bromo-6-methylbenzothiazole;
2-Bromo-6-fluorobenzothiazole;
2-Bromo-6-chlorobenzothiazole;
1-(2-Bromo-benzothiazol-6-yl)-ethanone;
2-Bromo-7-chloro-6-fluoro-benzothiazole;
7-Chloro-6-fluoro-2-thiophen-3-yl-benzothiazole; or 7-Bromo-benzothiazole.

12. A compound which is:
4-(Benzothiazol-2-yl)-thiophene-2-sulfonic acid amide;
4-(6-Nitro-benzothiazol-2-yl)-thiophene-2-sulfonic acid amide;
4-(6-Methyl-benzothiazol-2-yl)-thiophene-2-sulfonic acid amide;
4-(6-Fluoro-benzothiazol-2-yl)-thiophene-2-sulfonic acid amide;
4-(6-Chloro-benzothiazol-2-yl)-thiophene-2-sulfonic acid amide;
4-(6-Acetyl-benzothiazol-2-yl)-thiophene-2-sulfonic acid amide;
4-[7-(4-Fluoro-phenyl)-benzothiazol-2-yl]-thiophene-2-sulfonic acid amide;
4-(7-Chloro-6-fluoro-benzothiazol-2-yl)-thiophene-2-sulfonic acid amide;
4-Benzothiazol-2-yl-thiophene-2-sulfonic acid (2-fluoro-ethyl)-amide;
4-Benzothiazol-2-yl-thiophene-2-sulfonic acid thiazol-2-ylamide;
4-Benzothiazol-2-yl-thiophene-2-sulfonic acid (4,5-dimethyl-thiazol-2-yl)-amide;
4-Benzothiazol-2-yl-thiophene-2-sulfonic acid (3-hydroxy-pyridin-2-yl)-amide;
4-Benzothiazol-2-yl-thiophene-2-sulfonic acid (6-morpholin-4-yl-pyridin-3-yl)-amide;
4-Benzothiazol-2-yl-thiophene-2-sulfonic acid (pyridin-2-ylmethyl)-amide;
[1-(4-Benzothiazol-2-yl-thiophene-2-sulfonyl)-pyrrolidin-2-yl]-methanol;
4-Benzothiazol-2-yl-thiophene-2-sulfonic acid hydroxyamide;
[2-(4-Benzothiazol-2-yl-thiophene-2-sulfonylamino)-thiazol-4-yl]-acetic acid ethyl ester: or
4-Benzothiazol-2-yl-thiophene-2-sulfonic acid (4-methyl-pyridin-2-yl)-amide.

13. A pharmaceutical formulation comprising as active ingredient a therapeutically effective amount of a compound according to any one of claims 1 to 12 in association with pharmaceutically acceptable carriers or diluents.

14. The pharmaceutical formulation according to claim 13 for use in the prevention and/or treatment of conditions associated cyclin-dependent kinase 5.

15. A compound as defined in any one of claims 1 to 12 for use in therapy.

16. Use of a compound according to any one of claims 1 to 12 in the manufacture of a medicament for prevention and/or treatment of conditions associated with cyclin-dependent kinase 5.

17. Use of a compound according to any one of claims 1 to 12 in the manufacture of a medicament for prevention and/or treatment of dementia, Alzheimer's Disease, Parkinson's Disease, Frontotemporal dementia Parkinson's Type, Parkinson dementia complex of Guam, HIV dementia, diseases with associated neurofibrillar tangle pathologies, progressive supranuclear palsy, and dementia pugilistica.

18. Use of a compound according to claim 17, wherein the disease is Alzheimer's Disease.

19. Use of a compound according to any one of claims 1 to 12 in the manufacture of a medicament for prevention and/or treatment of amyotrophic lateral sclerosis, corticobasal degeneration, Down syndrome, Huntington's Disease, postencephelatic parkinsonism, progressive supranuclear palsy, Pick's Disease, Niemann-Pick's Disease, stroke, head trauma and other chronic neurodegenerative diseases.

20. Use of a compound according to any one of claims 1 to 12 in the manufacture of a medicament for prevention and/or treatment of stroke or chronic drug abuse.

21. A method of prevention and/or treatment of conditions associated with cyclin-dependent kinase 5, comprising administrering to a mammal, including man in need of such prevention and/or treatment, a therapeutically effective amount of a compound of formula I as defined in any one of claims 1 to 12.

22. A method of prevention and/or treatment of dementia, Alzheimer's Disease, Parkinson's Disease, Frontotemporal dementia Parkinson's Type, Parkinson dementia complex of Guam, HIV dementia, diseases with associated neurofibrillar tangle pathologies, progressive supranuclear palsy, and dementia pugilistica, comprising administrering to a mammal, including man in need of such prevention and/or treatment, a therapeutically effective amount of a compound of formula I as defined in any one of claims 1 to 12.

23. The method according to claim 22, wherein the disease is Alzheimer's Disease.

24. A method of prevention and/or treatment of amyotrophic lateral sclerosis, corticobasal degeneration, Down syndrome, Huntington's Disease, postencephelatic parkinsonism, progressive supranuclear palsy, Pick's Disease, Niemann-Pick's Disease, stroke, head trauma and other chronic neurodegenerative diseases, comprising administrering to a mammal, including man in need of such prevention and/or treatment, a therapeutically effective amount of a compound of formula I as defined in any one of claims 1 to 12.

25. A method of prevention and/or treatment of stroke or chronic drug abuse, comprising administrering to a mammal, including man in need of such prevention and/or treatment, a therapeutically effective amount of a compound of formula I as defined in any one of claims 1 to 12.

26. A process for the preparation of a compound of formula I according to claim 1, wherein R, R¹, R², R³, R⁴, R⁵ are, unless specified otherwise, defined as in formula I of claim 1, 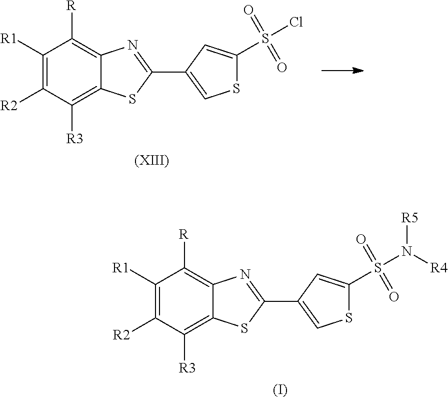
comprising amidating a compound of formula XIII with a suitable amine in the presence of a suitable solvent, to obtain a compound of formula I.
